(19) United States
(12) Patent Application Publication
(10) Pub. No.: US 2025/0005687 A1
(43) Pub. Date: Jan. 2, 2025

(54) RENEWABLE ENERGY USAGE WITHIN WIRELESS NETWORKS

(71) Applicant: RAKUTEN SYMPHONY, INC., Setagaya-ku, Tokyo (JP)

(72) Inventor: Manmeet Singh BHANGU, Indore (IN)

(21) Appl. No.: 18/276,131

(22) PCT Filed: Jul. 14, 2023
(86) PCT No.: PCT/US2023/027736

§ 371 Date: Aug. 7, 2023

(30) Foreign Application Priority Data
Feb. 3, 2023 (IN) 202321006920

(51) Int. Cl. G06Q 50/06; H04W 4/38

(52) U.S. Cl. CPC G06Q 50/06; H04W 4/38

(57) ABSTRACT

Provided are a system, method, and device for renewable energy usage within a network. According to embodiments, the system can include a memory storage storing computer-executable instructions, and at least one processor communicatively coupled to the memory storage, wherein the at least one processor is configured to execute the instructions to identify one or more network resources using a renewable energy source; and calculate an energy consumption value for a user using the identified one or more network resources.

### TECHNICAL FIELD

The present disclosure described herein relates to methods and systems for renewable energy usage within wireless networks.

### BACKGROUND

Renewable energy can generally be energy derived from natural sources that are replenished at a higher rate than they are consumed. For example, solar, wind, geothermal, hydropower, ocean, and bio-energy are such renewable energy sources that are continuously being replenished due to their inherent nature. In contrast, non-renewable energy can include fossil fuels, such as coal, oil, and gas, which can take millions of years to form and can further produce harmful greenhouse gas emissions, such as carbon dioxide.

Network service operators have recently set certain targets to reduce carbon intensity and their carbon footprint over the next few years with the ultimate goal of achieving net-zero emissions. While 5G New Radio (NR) offers improved energy-efficiency, new 5G use cases and the wider adoption of mmWave can result in an increased number of sites, base stations, and antennas, which may offset the foregoing energy efficiency gains if left unmitigated. Accordingly, operators have an interest in powering their networks using renewable energy sources to reduce emissions and enhance network efficiency. It is also important for operators to understand and track the proportion of energy consumed in their networks that is sourced from renewable sources, which can be made available to customers and authorized third parties.

Hence, what is needed is a system, process, and device that allows network service operators to identify the amount of renewable energy they utilize within their networks and to further allow its customers and subscribers to view their individual energy consumption usage, including each customer's usage that includes renewable energy sources.

### SUMMARY

Example embodiments of the present disclosure provide a system, a method, and a device that allows network service operators to identify the amount of renewable energy they utilize within their networks and to further allow its customers and subscribers to view their individual energy consumption usage, including each customer's usage that includes renewable energy sources. The present disclosure described herein can be cost-effective, utilizes minimal computing resources, and further helps to reduce the carbon footprint of network service providers/operators and its users. In addition, the present disclosure described herein can help to incentive network service operators to implement network resources, components, and functions that are more energy efficient and rely on renewable energy sources. Moreover, the present disclosure described herein can ensure transparency and accountability for network service operators that use renewable energy usage versus non-renewable energy sources, thereby further improving customer satisfaction and/or abiding by certain regulations. In addition, the present disclosure described herein allows a convenient method for customers of a network service provider to manage and view their energy usage such that they can make certain changes to their network usage behavior in order to maximize energy efficiency, costs, and the use of renewable energy sources, among other advantages and technical improvements.

According to some embodiments, a system is provided. The system that can include a memory storage storing computer-executable instructions; and at least one processor communicatively coupled to the memory storage, wherein the at least one processor is configured to execute the instructions to identify one or more network resources using a renewable energy source; and calculate an energy consumption value for a user using the identified one or more network resources.

According to some embodiments, a method is provided. The method performed by at least one processor, can include identifying one or more network resources using a renewable energy source; and calculating an energy consumption value for a user using the identified one or more network resources.

According to some embodiments, a non-transitory computer-readable recording medium is provided. The non-transitory computer-readable recording medium having recorded thereon instructions executable by at least one processor to cause the at least one processor to perform a method including identifying one or more network resources using a renewable energy source; and calculating an energy consumption value for a user using the identified one or more network resources.

### BRIEF DESCRIPTION OF THE DRAWINGS

Features, advantages, and significance of exemplary embodiments of the disclosure will be described below with reference to the accompanying drawings, in which like signs denote like elements, and wherein:

FIG. 1 illustrates a diagram of a general system architecture of the present disclosure described herein, according to one or more embodiments; and

FIG. 2 illustrates a process flow diagram for a method of the present disclosure described herein, according to one or more embodiments.

### DETAILED DESCRIPTION

The following detailed description of example embodiments refers to the accompanying drawings. The same reference numbers in different drawings may identify the same or similar elements.

The foregoing disclosure provides illustrations and descriptions, but is not intended to be exhaustive or to limit the implementations to the precise form disclosed. Modifications and variations are possible in light of the above disclosure or may be acquired from practice of the implementations. Further, one or more features or components of one embodiment may be incorporated into or combined with another embodiment (or one or more features of another embodiment). Additionally, in the flowcharts and descriptions of operations provided below, it is understood that one or more operations may be omitted, one or more operations may be added, one or more operations may be performed simultaneously (at least in part), and the order of one or more operations may be switched.

It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware may be designed to implement the systems and/or methods based on the description herein.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Where only one item is intended, the term “one” or similar language is used. Also, as used herein, the terms “has,” “have,” “having,” “include,” “including,” or the like are intended to be open-ended terms. Further, the phrase “based on” is intended to mean “based, at least in part, on” unless explicitly stated otherwise. Furthermore, expressions such as “at least one of [A] and [B]” or “at least one of [A] or [B]” are to be understood as including only A, only B, or both A and B.

Reference throughout this specification to “one embodiment,” “an embodiment,” “non-limiting exemplary embodiment,” or similar language means that a particular feature, structure, or characteristic described in connection with the indicated embodiment is included in at least one embodiment of the present solution. Thus, the phrases “in one embodiment”, “in an embodiment,” “in one non-limiting exemplary embodiment,” and similar language throughout this specification may, but do not necessarily, all refer to the same embodiment.

Furthermore, the described features, advantages, and characteristics of the present disclosure may be combined in any suitable manner in one or more embodiments. One skilled in the relevant art will recognize, in light of the description herein, that the present disclosure can be practiced without one or more of the specific features or advantages of a particular embodiment. In other instances, additional features and advantages may be recognized in certain embodiments that may not be present in all embodiments of the present disclosure.

In one implementation of the present disclosure described herein, a display page may include information residing in the computing device's memory, which may be transmitted from the computing device over a network to a database center and vice versa. The information may be stored in memory at each of the computing device, a data storage resided at the edge of the network, or on the servers at the database centers. A computing device or mobile device may receive non-transitory computer readable media, which may contain instructions, logic, data, or code that may be stored in persistent or temporary memory of the mobile device, or may somehow affect or initiate action by a mobile device. Similarly, one or more servers may communicate with one or more mobile devices across a network, and may transmit computer files residing in memory. The network, for example, can include the Internet, wireless communication network, or any other network for connecting one or more mobile devices to one or more servers.

Any discussion of a computing, user device, user equipment, mobile device may also apply to any type of networked device, including but not limited to mobile devices and phones such as cellular phones (e.g., any “smart phone”), a personal computer, server computer, or laptop computer; personal digital assistants (PDAs); a roaming device, such as a network-connected roaming device; a wireless device such as a wireless email device or other device capable of communicating wireless with a computer network; or any other type of network device that may communicate over a network and handle electronic transactions. Any discussion of any mobile device mentioned may also apply to other devices, such as devices including short-range ultrahigh frequency (UHF) device, near-field communication (NFC), infrared (IR), and Wi-Fi functionality, among others.

Phrases and terms similar to “software”, “application”, “app”, and “firmware” may include any non-transitory computer readable medium storing thereon a program, which when executed by a computer, causes the computer to perform a method, function, or control operation.

Phrases and terms similar to “network” may include one or more data links that enable the transport of electronic data between computer systems and/or modules. When information is transferred or provided over a network or another communications connection (either hardwired, wireless, or a combination of hardwired or wireless) to a computer, the computer uses that connection as a computer-readable medium. Thus, by way of example, and not limitation, computer-readable media can also include a network or data links which can be used to carry or store desired program code means in the form of computer-executable instructions or data structures and which can be accessed by a general purpose or special purpose computer.

Phrases and terms similar to “portal” or “terminal” may include an intranet page, internet page, locally residing software or application, mobile device graphical user interface, or digital presentation for a user. The portal may also be any graphical user interface for accessing various modules, components, features, options, and/or attributes of the present disclosure described herein. For example, the portal can be a web page accessed with a web browser, mobile device application, or any application or software residing on a computing device.

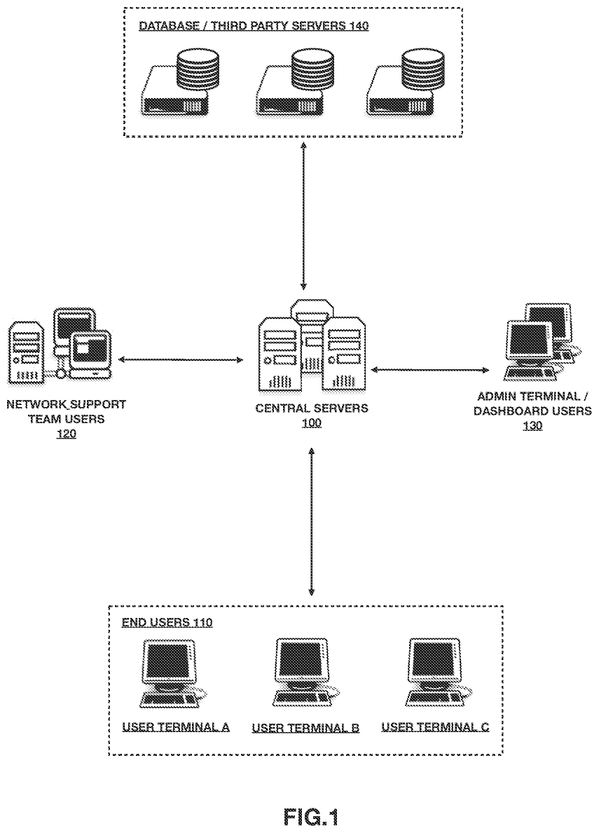

FIG. 1 illustrates a diagram of a general network architecture according to one or more embodiments. Referring to FIG. 1, end users 110, network support team users 120, and admin terminal/dashboard users 130 (collectively referred to herein as users 110, 120, and 130) can be in bi-directional communication over a secure network with central servers or application servers 100 according to one or more embodiments. In addition, users 110, 120, 130 may also be in direct bi-directional communication with each other via the network system of the present disclosure described herein according to one or more embodiments. Here, users 110 can be any type of customer, subscriber, network service provider agent, or vendor, among others, of a network or telecommunication service provider, such as users operating computing devices and user terminals A, B, and C. Each of users 110 can communicate with servers 100 via their respective terminals or portals. Users 120 can include application development members or support agents of the network service provider or operator for developing, integrating, and monitoring databases, front end systems and services, network traffic, and provisioning traffic, among others, including assisting, scheduling, and modifying network events, and providing support services to end users 110. Admin terminal/dashboard users 130 may be any type of user with access privileges for accessing a dashboard or management portal of the present disclosure described herein, wherein the dashboard portal can provide various user tools, GUI information, maps, open/closed/pending support tickets, graphs, customer support options, network status and performance, and KPIs, among others. It is contemplated within the scope of the present disclosure described herein that any of users 110 and 120 may also access the admin terminal/dashboard 130 of the present disclosure described herein.

Still referring to FIG. 1, central servers 100 of the present disclosure described herein according to one or more embodiments can be in further bi-directional communication with database/third party servers 140, which may also include users. Here, servers 140 can include renewable energy sources, convention energy sources, utility providers, energy consumption management entities for the network service provider and/or its customers, among others. In addition, servers 140 can include servers and databases for captured, collected, or aggregated data, such as current, real-time, and past network related historical and KPI data which may be stored thereon and retrieved therefrom for network analysis, energy consumption calculations (including energy from renewable sources), artificial intelligence (AI) processing, neural network models, machine learning, predictions, and simulations by servers 100. However, it is contemplated within the scope of the present disclosure described herein that the database migration system and method of the present disclosure described herein can include any type of general network architecture. Still referring to FIG. 1, one or more of servers or terminals of elements 100-140 may include a personal computer (PC), a printed circuit board including a computing device, a minicomputer, a mainframe computer, a microcomputer, a telephonic computing device, a wired/wireless computing device (e.g., a smartphone, a personal digital assistant (PDA)), a laptop, a tablet, a smart device, a wearable device, or any other similar functioning device.

In some embodiments, as shown in FIG. 1, one or more servers, terminals, and users 100-140 may include a set of components, such as a processor, a memory, a storage component, an input component, an output component, a communication interface, and a JSON UI rendering component. The set of components of the device may be communicatively coupled via a bus.

The bus may include one or more components that permit communication among the set of components of one or more of servers or terminals of elements 100-140. For example, the bus may be a communication bus, a cross-over bar, a network, or the like. The bus may be implemented using single or multiple (two or more) connections between the set of components of one or more of servers or terminals of elements 100-140. The disclosure is not limited in this regard.

One or more of servers or terminals of elements 100-140 may include one or more processors. The one or more processors may be implemented in hardware, firmware, and/or a combination of hardware and software. For example, the one or more processors may include a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), a general purpose single-chip or multi-chip processor, or other programmable logic device, discrete gate or transistor logic, discrete hardware components, or any combination thereof designed to perform the functions described herein. A general purpose processor may be a microprocessor, or any conventional processor, controller, microcontroller, or state machine. The one or more processors also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function.

The one or more processors may control overall operation of one or more of servers or terminals of elements 100-140 and/or of the set of components of one or more of servers or terminals of elements 100-140 (e.g., memory, storage component, input component, output component, communication interface, rendering component).

One or more of servers or terminals of elements 100-140 may further include memory. In some embodiments, the memory may include a random access memory (RAM), a read only memory (ROM), an electrically erasable programmable ROM (EEPROM), a flash memory, a magnetic memory, an optical memory, and/or another type of dynamic or static storage device. The memory may store information and/or instructions for use (e.g., execution) by the processor.

A storage component of one or more of servers or terminals of elements 100-140 may store information and/or computer-readable instructions and/or code related to the operation and use of one or more of servers or terminals of elements 100-140. For example, the storage component may include a hard disk (e.g., a magnetic disk, an optical disk, a magneto-optic disk, and/or a solid state disk), a compact disc (CD), a digital versatile disc (DVD), a universal serial bus (USB) flash drive, a Personal Computer Memory Card International Association (PCMCIA) card, a floppy disk, a cartridge, a magnetic tape, and/or another type of non-transitory computer-readable medium, along with a corresponding drive.

One or more of servers or terminals of elements 100-140 may further include an input component. The input component may include one or more components that permit one or more of servers and terminals 100-140 to receive information, such as via user input (e.g., a touch screen, a keyboard, a keypad, a mouse, a stylus, a button, a switch, a microphone, a camera, and the like). Alternatively or additionally, the input component may include a sensor for sensing information (e.g., a global positioning system (GPS) component, an accelerometer, a gyroscope, an actuator, and the like).

An output component any one or more of servers or terminals of elements 100-140 may include one or more components that may provide output information from the device 100 (e.g., a display, a liquid crystal display (LCD), light-emitting diodes (LEDs), organic light emitting diodes (OLEDs), a haptic feedback device, a speaker, and the like).

One or more of servers or terminals of elements 100-140 may further include a communication interface. The communication interface may include a receiver component, a transmitter component, and/or a transceiver component. The communication interface may enable one or more of servers or terminals of elements 100-140 to establish connections and/or transfer communications with other devices (e.g., a server, another device). The communications may be enabled via a wired connection, a wireless connection, or a combination of wired and wireless connections. The communication interface may permit one or more of servers or terminals of elements 100-140 to receive information from another device and/or provide information to another device. In some embodiments, the communication interface may provide for communications with another device via a network, such as a local area network (LAN), a wide area network (WAN), a metropolitan area network (MAN), a private network, an ad hoc network, an intranet, the Internet, a fiber optic-based network, a cellular network (e.g., a fifth generation (5G) network, sixth generation (6G) network, a long-term evolution (LTE) network, a third generation (3G) network, a code division multiple access (CDMA) network, and the like), a public land mobile network (PLMN), a telephone network (e.g., the Public Switched Telephone Network (PSTN)), or the like, and/or a combination of these or other types of networks. Alternatively or additionally, the communication interface may provide for communications with another device via a device-to-device (D2D) communication link, such as FlashLinQ, WiMedia, Bluetooth, ZigBee, Wi-Fi, LTE, 5G, and the like. In other embodiments, the communication interface may include an Ethernet interface, an optical interface, a coaxial interface, an infrared interface, a radio frequency (RF) interface, or the like. It is understood that other embodiments are not limited thereto, and may be implemented in a variety of different architectures (e.g., bare metal architecture, any cloud-based architecture or deployment architecture such as Kubernetes, Docker, OpenStack, etc.).

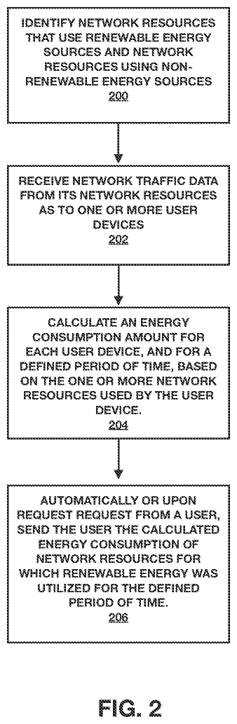

FIG. 2 illustrates a process flow for renewable energy usage within a wireless network system, method, and device of the present disclosure described herein according to one or more exemplary embodiments. In particular, the general process can begin as step 200. At step 200, a network service provider or operator can identify certain network resources that use renewable energy sources and certain network resources that use non-renewable energy sources. As an example, such network resources can be both physical and virtual components/elements and operations/functions within or associated with the operator's network systems and infrastructure, such as within a 5G network. For example, such physical and virtual components can include base stations, antennas, routers, switches, hubs, bridges, gateways, modems, repeaters, access points, databases, virtual machines, and various hardware, software, and cloud-based systems and computing devices. In addition, such network resources can include various network functions, such as in the case of a 5G network via 3GPP, Radio Access Network (RAN), core transport networks, or any type of network architecture, including a Access and Mobility Management Function (AMF), Session Management Function (SMF), Application Function (AF), User Plane Function (UPF), Network Repository Function (NRF), Policy Control Function (PCF), Binding Support Function (BSF), Network Slicing Selection Function (NSSF), Service Communication Proxy (SCP), Unified Data Management (UDM), User Data Repository (UDR), Authentication Server Function (AUSF), and Network Data Analytics Function (NWDAF), Network Function Virtualization (NFV), beamforming, distributed computing power functions, among others.

Still referring to step 200 of FIG. 2, in other embodiments, certain network resources may also be certified or include certifications, such as renewable energy certificates (RECs) that certify (such as from a third party or energy/utility company) that a particular network resource is net zero-emissions (uses only renewable energy sources) or that the network resource uses a certain percentage of renewable energy. As an example, an operator's base station may be certified as being powered by only renewable energy sources. Once the network resources have been identified as using renewable energy sources, then the process can proceed to step 202. At step 202, the network service operator can receive network traffic for each user device, computing device, or user equipment that has connected and/or communicated with any of its network resources. Here, the operator can also identify if the user device that used its network resources is a customer or subscriber of the operator (or any other network service provider affiliated with the operator). For example, each user device may include a device ID, any type of unique identifier, or any other subscriber identification system, function, or module. Once the system has analyzed the traffic data and identified and categorized each user device, including each device's usage of the operator's network resources, then the process can proceed to step 204.

In addition, it is contemplated within the scope of the present disclosure described herein that the user, customer, or subscriber of the network service provider can select which types of renewable energy sources it wishes to use, such as wind, solar, hydropower, geothermal, or bio-energy, or a combination of one or more of the foregoing. As an example, if the user selects wind and solar, then the network system of the present disclosure described herein can be configured to allow the user (via its user device(s)/user equipment) to only use certain network resources that are powered by the selected renewable energy sources, such as base stations that are powered by only wind and solar, and not geothermal. Alternatively, the user may select an option such that allows it to use network resources or functions of the operator that only use renewable energy sources altogether. In such an example, if there are certain network resources within the operator's network that use non-renewable sources, then the system can automatically direct or re-direct the user device's communications and interactions with its network to only network resources that use renewable energy sources, such as base stations, routers, virtual machines, and switches powered only by renewable energy sources. As another example, according to the 3GPP guidance, the quality of the connection must not be deteriorated if a user has to use an alternative source of energy, wherein it is encouraged to use renewable energy but not at the cost of performance.

As another example, the user can also be notified that certain network access or operations may be restricted if the user selects certain renewable energy sources or all renewable energy sources, and no non-renewable energy sources (e.g., network coverage being 80% if only renewable energy sources are used, as opposed to 100% if both renewable and non-renewable energy sources are used). For example, if the user selected only renewable energy sources, then a base station in a user device's path that uses non-renewable energy sources (e.g., fossil fuels) can refuse to accept, deny, or block a handoff procedure between another base station that does use renewable energy sources. Thus, in the foregoing example, for that period of time that the user device is within the vicinity of only the base station that uses non-renewable sources, then network access can be limited for the user device until it is within the vicinity of another base station that does use a renewable energy source, among other exemplary embodiments.

Still referring to FIG. 2, at step 204, the system can calculate an energy consumption amount, value, or percentage for each user device. Here, the energy consumption amount can be calculated for a defined period of time and further based on the one or more network resources used by the user device, including the power/bandwidth requirements and duration of such use, among other factors. In addition, the energy consumption amount may be based on renewable energy sources used by the user relative to a percentage of non-renewable energy sources used by the user. As an example, the system may be able to calculate the user device's usage of one or more neighboring base stations for a given period of time (and whether those base stations are using renewable or non-renewable energy sources) and further identify network resources (such as routers or other nodes) that use renewable or non-renewable energy sources in connection with the base stations. Based on the foregoing, the system can calculate that for a defined period of time, such as within one year, week, or a day, the user device's (including the user/subscriber/customer) energy consumption of the operator's network and its network resources came from a certain percentage of renewable sources, such as 60%. Once the user's energy consumption has been calculated in step 204, then the process may proceed to step 206.

In another example, the system of the present disclosure described herein can calculate energy usage of a virtual machine, wherein a percentage of the usage is associated with a renewable energy source. Such a calculation process can include each customer being designated a virtual machine (e.g., base station, router, etc.) within the operator's network. Further, if there are two similar network functions (NF), such as NF1 and NF, NF1 can be a virtual machine (VM) in a data center running on renewable energy while NF2 (also a VM) can be running on another server that uses conventional energy. Accordingly, if the NF's are realized as virtual machines, they can be simple to move and migrate them between servers based on whether they are using renewable energy sources or conventional energy sources.

Still referring to FIG. 2, at step 206, the system may automatically, periodically (such as based on a schedule), or manually upon a request of a user, provide to the user the user's energy usage consumption value, amount, or percentage for a defined period of time, including the amount of energy, costs, and the like stemming from the user's use of network resources or functions that use renewable energy sources. In particular, such usage may be sent based on a request from the user for such information, sent automatically or via a defined schedule by the system of the present disclosure described herein. In addition, it is contemplated within the scope of the present disclosure described herein that such information may be sent as a report from the network service operator, or as periodic updates and notifications to the user via email, SMS, wireless messaging, or the like. With respect to the reports, in one embodiment, the system of the present disclosure described herein can include a detailed breakdown of the user's activity, including but not limited to: the specific network resources used (and whether they used renewable sources), costs associated with usage of the network resources, the types of renewable energy sources used, a network slice that operates on renewable energy, the user's usage per network resource function, the duration of such use, reporting renewable energy certificates (RECs) in connection with the used network resources or functions, power requirements of such use, the user's percentage contribution in reducing emissions, and various suggestions for further reducing emissions, among others. Such suggestions can include the user changing certain network usage behaviors and avoiding use of network resources in certain areas or neighborhoods that use non-renewable energy sources, among others. In one example, the user/customer may be able to use such information in order to qualify for certain tax credits (or any other incentive, promotion, or award) from a government, public, or private institution for using renewable energy sources. In addition, the network service provider may have a process in place that can certify the foregoing reports for such tax reporting purposes.

Another non-limiting exemplary embodiment of the present disclosure described herein can include, subject to an operator's policy, its network system can acquire renewable energy usage information of the network functions serving the customer and expose this information to the customer or authorized third parties. For example, third parties may be able to use such information to further optimize the operator's network and further manage a user/customer's energy consumption usage.

Another non-limiting exemplary embodiment of the present disclosure described herein can include, subject to operator's policy and consent by the vertical customer, its network system can provide a customer a network slice that operates on renewable energy and make this information accessible to the customer and designated third parties.

Another non-limiting exemplary embodiment of the present disclosure described herein can include, subject to operator's policy and consent by the vertical customer, the 5G system shall have the capability to supply individualized renewable energy charging services to the customer.

Another non-limiting exemplary embodiment of the present disclosure described herein can include the operator's network system providing real-time monitoring and reporting of renewable energy usage, including the energy source and usage per network function, to ensure transparency and accountability for renewable energy usage.

Another non-limiting exemplary embodiment of the present disclosure described herein can include the operator's network system providing the ability for customers to select their preferred renewable energy source (e.g., wind, solar, hydropower, etc.) for their network operations, subject to operator's policy and available energy sources.

Another non-limiting exemplary embodiment of the present disclosure described herein can include the operator's network system having the capability to securely and accurately track and report renewable energy certificates (RECs) to support the customer's renewable energy procurement and management efforts.

Another non-limiting exemplary embodiment of the present disclosure described herein can include the operator's network system providing customers with a detailed breakdown of renewable energy mix and costs, allowing them to track and manage their energy consumption effectively. For example, in some regions, solar and wind may be preferred over nuclear energy.

Another non-limiting exemplary embodiment of the present disclosure described herein can include, subject to an operator's policy, a 5G network system shall enable the operation of a dedicated network above a minimum ratio of renewable energy as requested by an authorized third party.

Another non-limiting exemplary embodiment of the present disclosure described herein can include, subject to the operator's policy, the 5G network system shall be able to provide to a third party the reporting of the ratio of renewable energy used to provide dedicated communication service to the third party on a periodic basis.

It is understood that the specific order or hierarchy of blocks in the processes/flowcharts disclosed herein is an illustration of example approaches. Based upon design preferences, it is understood that the specific order or hierarchy of blocks in the processes/flowcharts may be rearranged. Further, some blocks may be combined or omitted. The accompanying method claims present elements of the various blocks in a sample order, and are not meant to be limited to the specific order or hierarchy presented.

Some embodiments may relate to a system, a method, and/or a computer readable medium at any possible technical detail level of integration. Further, one or more of the above components described above may be implemented as instructions stored on a computer readable medium and executable by at least one processor (and/or may include at least one processor). The computer readable medium may include a computer-readable non-transitory storage medium (or media) having computer readable program instructions thereon for causing a processor to carry out operations.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may include copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program code/instructions for carrying out operations may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a standalone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects or operations.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer readable media according to various embodiments. In this regard, each block in the flowchart or block diagrams may represent a module, microservice(s), segment, or portion of instructions, which includes one or more executable instructions for implementing the specified logical function(s). The method, computer system, and computer readable medium may include additional blocks, fewer blocks, different blocks, or differently arranged blocks than those depicted in the Figures. In some alternative implementations, the functions noted in the blocks may occur out of the order noted in the Figures. For example, two blocks shown in succession may, in fact, be executed concurrently or substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware may be designed to implement the systems and/or methods based on the description herein.

Various further respective aspects and features of embodiments of the present disclosure may be defined by the following items:

Item [1]: A system including a memory storage storing computer-executable instructions; and at least one processor communicatively coupled to the memory storage, wherein the at least one processor is configured to execute the instructions to identify one or more network resources using a renewable energy source; and calculate an energy consumption value for a user using the identified one or more network resources.

Item [2]: The system of item [1], wherein the at least one processor is configured to execute the instructions to send the energy consumption value to the user.

Item [3]: The system of item [2], wherein the energy consumption value is sent to the user based on the user's request.

Item [4]: The system of item [2], wherein the energy consumption value is sent to the user automatically or based on a defined schedule.

Item [5]: The system of item [2], wherein the sent energy consumption value is sent within a report, wherein the report includes at least one of: a cost associated with usage of the network resources, renewable energy sources, a network slice that operates on renewable energy, usage per network resource function, or tax savings or tax credits associated with usage of the network resources.

Item [6]: The system of item [1], wherein the at least one processor is configured to execute the instructions to receive a selection for one or more types of renewable energy sources from the user.

Item [7]: The system of items [1]-[6], wherein the one or more types of renewable energy sources include one or more of: wind, solar, hydropower, ocean, geothermal, and bio-energy.

Item [8]: The system of item [1], wherein the calculated energy consumption value is based on a defined period of time.

Item [9]: The system of item [8], wherein the calculated energy consumption value is further based an amount of power utilized by the user in connection with one or more network resources.

Item [10]: The system of item [1], wherein the calculated energy consumption value is based on a percentage of renewable energy sources used by the user relative to a percentage of non-renewable energy sources used by the user.

Item [11]: A method, performed by at least one processor, including identifying one or more network resources using a renewable energy source; and calculating an energy consumption value for a user using the identified one or more network resources.

Item [12]: The method of item [11], further including sending the energy consumption value to the user.

Item [13]: The method of item [12], wherein the energy consumption value is sent to the user based on the user's request.

Item [14]: The method of item [12], wherein the energy consumption value is sent to the user automatically or based on defined schedule.

Item [15]: The method of item [12], wherein the sent energy consumption value is sent within a report, wherein the report includes at least one of: costs associated with usage of the network resources, renewable energy sources, a network slice that operates on renewable energy, or usage per network resource function.

Item [16]: The method of item [11], further including receiving a selection for one or more types of renewable energy sources from the user.

Item [17]: The method of items [11]-[16], wherein the one or more types of renewable energy sources include one or more of: wind, solar, hydropower, ocean, geothermal, and bio-energy.

Item [18]: The method of item [11], wherein the calculated energy consumption value is based on a defined period of time.

Item [19]: The method of item [18], wherein the calculated energy consumption value is further based on an amount of power utilized by the user in connection with the one or more network resources.

Item [20]: A non-transitory computer-readable recording medium having recorded thereon instructions executable by at least one processor to cause the at least one processor to perform a method including identifying one or more network resources using a renewable energy source; and calculating an energy consumption value for a user using the identified one or more network resources.

It can be understood that numerous modifications and variations of the present disclosure are possible in light of the above teachings. It will be apparent that within the scope of the appended clauses, the present disclosures may be practiced otherwise than as specifically described herein.

What is claimed is:

1. A system comprising:
a memory storage storing computer-executable instructions; and
at least one processor communicatively coupled to the memory storage, wherein the at least one processor is configured to execute the instructions to:
identify one or more network resources using a renewable energy source; and
calculate an energy consumption value for a user using the identified one or more network resources.

2. The system of claim 1, wherein the at least one processor is configured to execute the instructions to:
send the energy consumption value to the user.

3. The system of claim 2, wherein the energy consumption value is sent to the user based on the user's request.

4. The system of claim 2, wherein the energy consumption value is sent to the user automatically or based on a defined schedule.

5. The system of claim 2, wherein the sent energy consumption value is sent within a report, wherein the report comprises at least one of: a cost associated with usage of the network resources, renewable energy sources, a network slice that operates on renewable energy, usage per network resource function, or tax savings or tax credits associated with usage of the network resources.

6. The system of claim 1, wherein the at least one processor is configured to execute the instructions to:
receive a selection for one or more types of renewable energy sources from the user.

7. The system of claim 6, wherein the one or more types of renewable energy sources are comprised of one or more of: wind, solar, hydropower, ocean, geothermal, and bio-energy.

8. The system of claim 1, wherein the calculated energy consumption value is based on a defined period of time.

9. The system of claim 8, wherein the calculated energy consumption value is further based an amount of power utilized by the user in connection with one or more network resources.

10. The system of claim 1, wherein the calculated energy consumption value is based on a percentage of renewable energy sources used by the user relative to a percentage of non-renewable energy sources used by the user.

11. A method, performed by at least one processor, comprising:
identifying one or more network resources using a renewable energy source; and
calculating an energy consumption value for a user using the identified one or more network resources.

12. The method of claim 11, further comprising:
sending the energy consumption value to the user.

13. The method of claim 12, wherein the energy consumption value is sent to the user based on the user's request.

14. The method of claim 12, wherein the energy consumption value is sent to the user automatically or based on a defined schedule.

15. The method of claim 12, wherein the sent energy consumption value is sent within a report, wherein the report comprises at least one of: a cost associated with usage of the network resources, renewable energy sources, a network slice that operates on renewable energy, or usage per network resource function.

16. The method of claim 11, further comprising:
receiving a selection for one or more types of renewable energy sources from the user.

17. The method of claim 16, wherein the one or more types of renewable energy sources are comprised of one or more of: wind, solar, hydropower, ocean, geothermal, and bio-energy.

18. The method of claim 11, wherein the calculated energy consumption value is based on a defined period of time.

19. The method of claim 18, wherein the calculated energy consumption value is further based on an amount of power utilized by the user in connection with the one or more network resources.

20. A non-transitory computer-readable recording medium having recorded thereon instructions executable by at least one processor to cause the at least one processor to perform a method comprising:
identifying one or more network resources using a renewable energy source; and
calculating an energy consumption value for a user using the identified one or more network resources.
